(19) United States
(12) Patent Application Publication
(10) Pub. No.: US 2019/0336039 A1
(43) Pub. Date: Nov. 7, 2019

(54) APPARATUS AND METHOD FOR IDENTIFYING A COUGHING EVENT

(71) Applicants: University of Maryland, Baltimore, Baltimore, MD (US); University of Maryland, College Park, Collge Park, MD (US)

(72) Inventors: Stella HINES, Baltimore, MD (US); Lucia FERNANDEZ, Monroe, NJ (US); Jessica BAER, Holladay, UT (US); Andrew KADISH, Pikesville, MD (US); Yousuf NAVED, North Potomac, MD (US); Farah VEJZAGIC, Baltimore, MD (US); Ian WHITE, Ellicott City, MD (US)

(73) Assignees: University of Maryland, Baltimore, Baltimore, MD (US); University of Maryland, College Park, College Park, MD (US)

(21) Appl. No.: 16/402,849

(22) Filed: May 3, 2019

Related U.S. Application Data
(60) Provisional application No. 62/666,293, filed on May 3, 2018.

(51) Int. Cl. A61B 5/08; A61B 5/11; A61B 5/0488; A61B 5/00

(52) U.S. Cl. CPC A61B 5/0823; A61B 5/1107; A61B 5/113; A61B 5/7264; A61B 5/0488

(57) ABSTRACT

Provided is a method for identifying a coughing event, the method including obtaining, from a sensor included in a monitoring device worn by a patient, a plurality of digital voltage values, analyzing the plurality of digital voltage values to identify a physiological event based on (i) a change in magnitude of the digital voltage values and (ii) a duration of the change in the magnitude of the digital voltage values, the change in the magnitude of the digital voltage values and the duration of the change in the magnitude of the digital voltage values representing a first point in a two-dimensional feature space, and categorizing the identified physiological event as the coughing event based on a threshold hyperplane existing in the two-dimensional feature space.

### BACKGROUND OF THE INVENTION

### 1. Field of the Invention

The subject matter of the present disclosure relates generally to a device configured to monitoring and identifying a cough event.

### 2. Description of the Related Art

Chronic cough is an extremely prevalent condition. In the United States, as recently as 2015, around one in ten people reported suffering from chronic cough to their local pulmonologists. To further emphasize its prevalence, chronic cough is recognized as one of the most common symptoms prompting medical visits by patients. This is in part due to the fact that chronic cough itself is a symptom of a wide variety of diseases such as pulmonary fibrosis, asthma, environmental exposure, chronic obstructive pulmonary disease (COPD), and many more. Additionally, many people suffer from idiopathic chronic cough, or chronic cough without a known cause, making a proper treatment more difficult to prescribe.

Pulmonologists, in order to treat patients effectively, need a method to monitor patient responses to a variety of treatments in real time in order to determine their efficacy. Pulmonologists commonly utilize conventional pulmonary function tests (PFTs), such as spirometry, lung volume measurement, diffusion capacity for carbon monoxide, and chest imaging tests, including chest computed tomography (CT) scans, to make objective assessments about the severity of the disease. Cough, however, is a symptom that may not be accurately assessed using these traditional tests. Though the mechanisms currently tested are affected by various diseases, the mechanisms provide no insight into the severity of a patient's cough. For example, when a patient with asthma undergoes spirometry, the results suggest the presence and severity of airflow obstruction, but do not characterize cough, which may be the symptom most affecting quality of life.

PFTs do not always provide a full, accurate picture of the severity of a disease and the symptoms affecting patients, limiting doctors' abilities to effectively monitor the progression of pulmonary diseases and the effects of prescribed medications. This leaves patients with life-altering cough potentially untreated.

Despite this issue, there are currently no effective methods or devices on the market to counteract it, leaving pulmonologists without crucial information necessary to diagnose and treat their patients with idiopathic cough.

Instead, pulmonologists are forced to rely on patient responses to a cough related questionnaire, which is taken during an in-person appointment. The current method used to monitor chronic cough is known as the Leicester Cough Questionnaire, which is a patient-completed survey for subjectively measuring quality of life with the chronic cough. However, the Leicester Cough Questionnaire fails to both provide quantifiable data pertaining to the patient's cough and monitor real time coughing episodes or responses to specific treatments. The survey's success ineffectively relies on patient memory and only provides the pulmonologist with subjective data.

By the time the patient is able to take the survey, the patient may have forgotten most of the details of their most recent coughing episodes. The survey itself also, even if accurate, only gives doctors subjective patient answers to the question set, and relationships between the survey and objective cough severity and sensitivity are hard to draw. Furthermore, subjective data and objective data recorded by audio devices and analyzed by professional cough counters do not correlate consistently, meaning that patients are not always able to accurately describe the severity and frequency of their cough, especially at night while sleeping. These shortcomings of the current system of monitoring idiopathic cough emphasize the importance of providing pulmonologists with a better alternative.

Various devices integrating sensors have been used in attempts to record and classify frequency and intensity of coughing events.

For example, a device utilizing an electromyograph (EMG) sensor has been used to monitor electrical muscle impulses during abdominal contraction, as well as closure and sudden opening of the glottis, both signs used to detect cough. Although the EMG sensor is fairly accurate and capable in its designation of what classifies as a cough, the EMG sensor is only truly effective when the patient was stationary. With too much movement, interference would render the sensor readings less viable, thereby making the EMG sensor less useful in the development of a device that is meant to be used in everyday life.

Another device utilizing a mechanomyogram (MMG) sensor has been used to monitor mechanical vibration from muscle contraction in patients during coughing episodes. The MMG sensor was placed on the abdominal region, and while the MMG sensor was capable of discriminating between a cough and normal breathing, the MMG sensor faced issues with interference when it came to speaking. Additional studies have found MMG sensors to be less effective than EMG sensors, and as such, the MMG sensor is not a feasible sensor for everyday life as the effectiveness of the sensor is limited to very specific and unrealistic conditions.

A device utilizing an accelerometer has also been explored. However, the analysis of the signals showed that the accelerometer had the lowest correlation and highest standard deviation with respect to the internal landmark movement and external optical marker movement, thereby indicating that the accelerometer is not an ideal choice for accurately monitoring cough.

A device using a microphone has also been attempted. In such a system, the microphone would record all sound for an extended period of time and then this sound data would be classified using a machine learning application, such as a neural network, to classify coughing events from other noises. The challenge of using a microphone lies in the differentiation of sounds that might appear similar. For example, it would be difficult to distinguish coughing from loud laughter.

Additionally, audio recordings often require some manual analysis, as is the case for a device that has been used in some clinical environments, the VitaloJak®, which requires manual analysis of a condensed 24-hour recording.

The Leicester Cough Monitor (LCM) also classifies audio data, and only requires minimal manual analysis for its software. Researchers attached a microphone connected to a recording device to a patient. They then downloaded the sound data to a computer and used an artificial neural network to classify coughing events from non-coughing events. Although the researchers reported high specificity and sensitivity of the device, the results were only validated over a period of two hours. Further, the sensitivity of the system dropped from 91 to 86% when a six-hour validation was performed, and no sensitivity data was provided for a longer period such as a twenty-four hour period

Another device utilizing a microphone is the PulmoTrack®. However, this device is focused on wheeze detection of patients in the pediatric critical care unit of hospitals. While it effectively detects wheezing, cough detection and classification are not its focus. Additionally, it is not ambulatory, therefore, the device is not able to be worn by a patient for everyday use.

Although microphones have been proven to be accurate in identifying coughing events, the use of a microphone to detect a cough also raises privacy concerns. For example, ambient sounds may be records through contact microphones, and many patients may find this an invasion of privacy. Accordingly, the use of a microphone to detect cough is not ideal for everyday life.

Another device known as the Lifeshirt was reliant on several sensors to detect cough: an electrocardiogram, an induction plethysmography, a 3-axis accelerometer, and a contact microphone placed on the throat. This device only achieved a sensitivity of 78.1%. Furthermore, the combination of all the different sensors did not significantly improve the performance of the device, in addition to raising the cost and complexity, leading to lower patient compliance.

As previously discussed, according to pulmonologists, chronic cough is very prevalent in the United States and can be caused by a range of illnesses, such as asthma, COPD, and various cancers. Additionally, many patients suffer from idiopathic chronic cough, for which a concrete cause has not yet been determined. Diagnostic tests currently available, however, fall short of characterizing cough. Instead, pulmonologists must use existing tests that measure different endpoints, such as airflow obstruction, to assess the severity of a disease, even though these diagnostic tests do not truly measure the symptom in question--chronic cough. Consequently, pulmonologists are unable to make accurate assessments about the efficacy of certain prescribed treatments.

Accordingly, there is a desire for a device configured to reliably identify and monitor a coughing event/chronic cough over long periods of time, therefore aiding pulmonologists in formulating an accurate diagnosis and tracking the efficacy of potential treatments

### SUMMARY OF THE INVENTION

An embodiment of the present invention is directed to a method for identifying a coughing event, the method comprising: obtaining, from a sensor included in a monitoring device worn by a patient, a plurality of digital voltage values; analyzing the plurality of digital voltage values to identify a physiological event based on (i) a change in magnitude of the digital voltage values and (ii) a duration of the change in the magnitude of the digital voltage values, the change in the magnitude of the digital voltage values and the duration of the change in the magnitude of the digital voltage values representing a first point in a two-dimensional feature space; and categorizing the identified physiological event as the coughing event based on a threshold hyperplane existing in the two-dimensional feature space.

Another embodiment of the present invention is directed to an apparatus for identifying a coughing event, the apparatus comprising: a processor; and a non-transitory memory having stored thereon executable instructions, which when executed cause the processor to perform operations including: obtaining, from a sensor included in a monitoring device worn by a patient, a plurality of digital voltage values; analyzing the plurality of digital voltage values to identify a physiological event based on (i) a change in magnitude of the digital voltage values and (ii) a duration of the change in the magnitude of the digital voltage values, the change in the magnitude of the digital voltage values and the duration of the change in the magnitude of the digital voltage values representing a first point in a two-dimensional feature space; and categorizing the identified physiological event as the coughing event based on a threshold hyperplane existing in the two-dimensional feature space.

Another embodiment of the present invention is directed to a system for identifying a coughing event, the system comprising: a monitoring device including a microcontroller and a piezoelectric sensor configured to output voltage values to the microcontroller in response to strain placed upon the piezoelectric sensor by a patient as a result of a physiological event, the microcontroller being capable of performing short-range wireless communication; and a computer device capable of performing the short-range wireless communication, wherein microcontroller converts the voltage values obtained from the piezoelectric sensor to digital voltage values, and outputs the digital voltage values to the computer device using the short-range wireless communication, wherein the computer device analyzes the plurality of digital voltage values to identify a physiological event based on (i) a change in magnitude of the digital voltage values and (ii) a duration of the change in the magnitude of the digital voltage values, the change in the magnitude of the digital voltage values and the duration of the change in the magnitude of the digital voltage values representing a first point in a two-dimensional feature space, wherein the threshold hyperplane is obtained by linear regression to optimize a margin between (i) a first class of one or more points in the two-dimensional feature space corresponding to a physiological event that is a coughing event and (ii) a second class of one or more points in the two-dimensional feature space corresponding to a physiological event that is not a coughing event, wherein the computer device categorizes the identified physiological event as the coughing event based on a threshold hyperplane existing in the two-dimensional feature space, and wherein when the identified physiological event is categorized as the coughing event, the computer device outputs an indication of the coughing event to a display.

### BRIEF DESCRIPTION OF THE DRAWINGS

In the drawings, like reference numbers generally indicate identical, functionally similar, and/or structurally similar elements.

FIG. 1 illustrates an example of a monitor device configured to be positioned on a patient's torso according to an embodiment of the present invention.

FIG. 2 illustrates one example of the components of the housing of the monitor device according to an embodiment of the present invention.

FIG. 3 illustrates one example of integration of a microcontroller and a sensor according to an embodiment of the present invention.

FIG. 4 illustrates a cough detection system 400 in which the monitor device communicates with a computer device according to an embodiment of the present invention.

FIG. 5 is a flow chart for analyzing data for determining whether or not a coughing event has occurred according to an embodiment of the invention.

FIG. 6 illustrates a graph depicting the results of the digital voltage values obtained during a coughing event during the initial testing of the monitor device according to an embodiment of the present invention.

FIGS. 7A and 7B illustrate graphs depicting the results of the digital voltage values during a yawning event and a deep breathing event, respectively, during the initial testing of the monitor device 100 according to an embodiment of the present invention.

FIG. 8 illustrates one example of display of the frequency of coughing events as a histogram according to an embodiment of the present invention.

FIG. 9 illustrates one example of the computer device allowing patient input at the time of a coughing event according to an embodiment of the present invention.

### DETAILED DESCRIPTION OF THE INVENTION

Provided is a device configured to identify and monitor a coughing event/chronic cough over long periods of time, therefore aiding pulmonologists in formulating an accurate diagnosis and tracking the efficacy of potential treatments. The device can be further configured to determine cough events, such as by having machining learning processes to analyze data using a support vector machine (SVM) machine learning algorithm, for example, to cluster coughing events together based on change in a digital voltage reading. Information obtained from the device can be transferred to an external device, such as a personal computer, mobile device, or dedicated display device. In one embodiment, the device can communicate with a mobile device using an application wirelessly via Bluetooth®. Thus, doctors can securely access patient data in real time. In another embodiment, the device and/or the app can receive input of patient notes to increase the accuracy of subjective cough data for review by pulmonologists. Thus, the device provides pulmonologists with the objective and subjective data needed to best treat their patients' chronic cough.

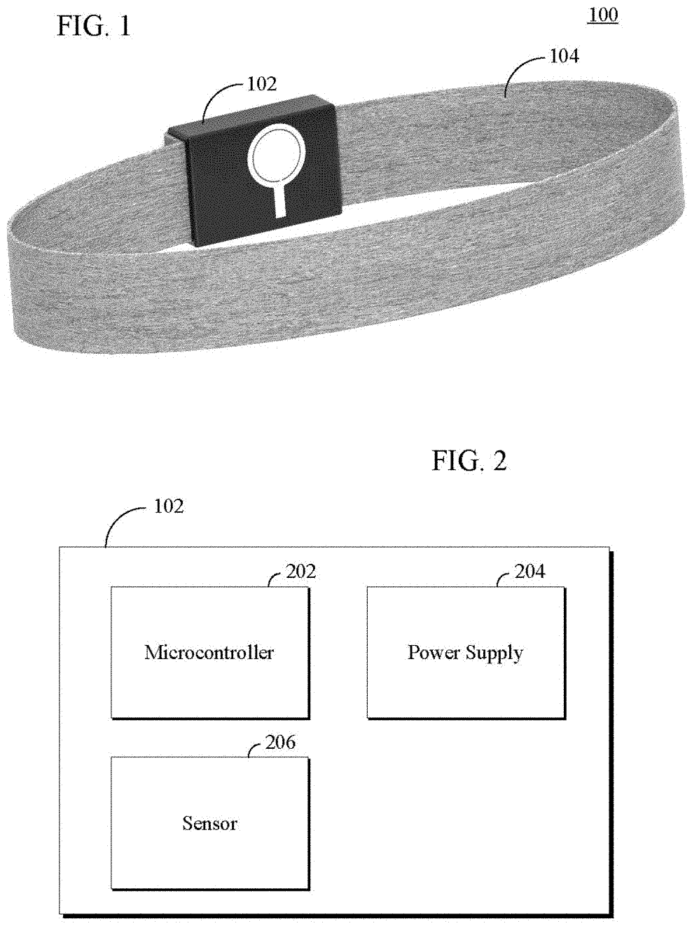

FIG. 1 illustrates an example of a monitor device 100 configured to be positioned on a patient's torso. As illustrated in FIG. 1, the monitor device 100 includes a housing 102 and an attachment portion 104. According to one embodiment, the housing is a small, 3D printed case, although the housing 102 may alternatively be a plastic injection molded electronics case.

The attachment portion 104 is a nylon webbing belt. However, the attachment portion 104 is not limited to this configuration, and the attachment portion 104 may be configured in another manner that would allow the monitor device 100 to be comfortably worn by the patient for an extended period of time.

Additionally, the monitor device 100 is not limited to a configuration including the attachment portion 104, that is, the monitor device 100 could alternatively be configured to be positioned on the patient via an adhesive patch.

FIG. 2 illustrates one example of the components of the housing 102 of the monitor device 100. As illustrated in FIG. 2, the housing 102 includes a microcontroller 202, a power supply 204, and a sensor 206. An example of the microcontroller is a Bluno Nano Arduino BLE (Bluetooth® low energy) microcontroller, which provides low power consumption and built-in Bluetooth® capabilities for data communication. However, the microcontroller 202 is not limited to this example, and other type of suitable controllers may be utilized. Additionally, data communication is not limited to Bluetooth®, and the microcontroller 202 may perform data communication by other wireless techniques such as Wi-Fi, cellular, and the like, and/or by wired techniques such as Ethernet, Universal Serial Bus (USB), and the like.

The monitor device 100 is configured to be comfortable to a patient such that the patient can wear the device for an extended period time. According to one embodiment, the monitor device 100 is intended to be tightly strapped to the upper abdominal region of the patient in order to obtain accurate readings from the sensor 204. In order to ensure maximum comfort, the monitor device 100 ideally weights less than 2 lbs, the sensor 204 is a flexible sensor capable of lying on an outside of the housing 102, and the size of the housing 102 is smaller than 5″×3″×1″. However, one of ordinary skill in the art would understand that monitor device 100 is not limited to such a configuration, and may be configured as necessary to ensure comfort an individual patient

The power supply 204 is chosen to allow the microcontroller to be remain fully active through a 24 hour period between charging of the monitor device 100. For example, in order to calculate battery requirements for the power supply 204, the typical current draw of the microcontroller 202 of less than 35 mA is rounded up to 40 mA to account for the current draw of sensor 203 and any fluctuations. Since the monitor device 100 is configured to operate for at least 24 hours between charging, the required battery capacity of the power supply 204 is calculated to be approximately 960 mAh. To meet this battery capacity, according to one embodiment of the present invention, the power supply 204 is implemented as a rechargeable lithium ion 3.7 V 2 Ah battery. However, the power supply 204 could be implemented as another battery type or other power supplies could be used in the monitor device 100.

For example, if the microcontroller 202 has a low power mode, the microcontroller 202 could be programmed to only be fully active when an input from the sensor 206 reaches a predetermined threshold, thereby allowing the microcontroller 202 to draw much less current, that is, only draw the predicted current of 35-45 mA when some physiological phenomenon such as a cough or yawn caused the input to cross that specified value. Configuration of the microcontroller 202 in this manner would allow the power supply 204 to be implemented using a battery with less storage capacity.

The sensor 206 is a piezoelectric strain sensor configured such that when the patient coughs, the patient's rib cage will push against the sensor 206 thereby changing the resistance of the piezoelectric circuit and creating an overall change in voltage.

A piezoelectric strain sensor was selected as the sensor 206 over other alternatives such as a microphone, an electromyography sensor, and an accelerometer due to a combination of ease of patient compliance, least amount of interference with daily life of a patient, and ability to differentiate between a couch and other events such as a laugh or deep breathing. However, the sensor 203 is not limited to only a piezoelectric strain sensor, and other types of sensors could be used which could satisfactory achieve the combination of ease of patient compliance, least amount of interference with daily life of a patient, and ability to differentiate between a couch and other events such as a laugh or deep breathing.

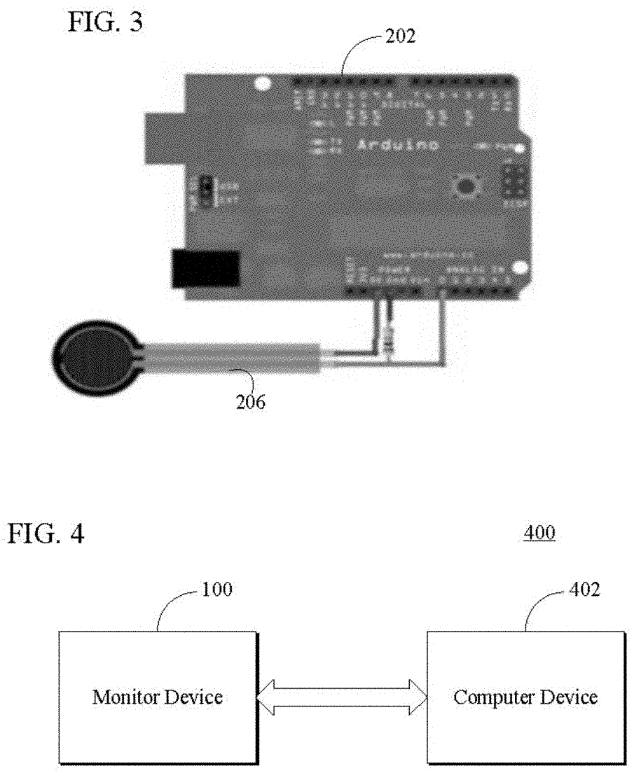

An example of the one embodiment of integration of the microcontroller 202 and the sensor 206 is illustrated in FIG. 3.

As illustrated in FIG. 3, a power output of the microcontroller 202 is connected to one terminal of the sensor 206, and a second terminal of the sensor 206 is connected to an analog input of the microcontroller 202, thereby allowing the microcontroller 202 to continuously receive voltage values from the sensor 206. By continuously receiving voltage values from the sensor 206, the microcontroller 202 is able detect a change in the voltage values from a change in the resistance of the piezoelectric circuit as a result of a push against the sensor 206. The microcontroller 202 has the ability to convert the voltage values received at the analog input into digital voltage values. As will be described later, these digital voltage values will be analyzed to determine whether or not a coughing event has occurred.

FIG. 4 illustrates an embodiment of a cough detection system 400 in which the monitor device 100 communicates with a computer device 402. According to one embodiment, the computer device 402 is a personal computer, dedicated display device, smartphone, or other mobile computer device capable of communicating with the monitor device 100. However, the computer device 402 is not limited to such a configuration.

According to one embodiment, the computer device 402 executes a computer program to communicate with the monitor device 100 to obtain data and perform analysis on the received data to determine whether or not a coughing event has occurred.

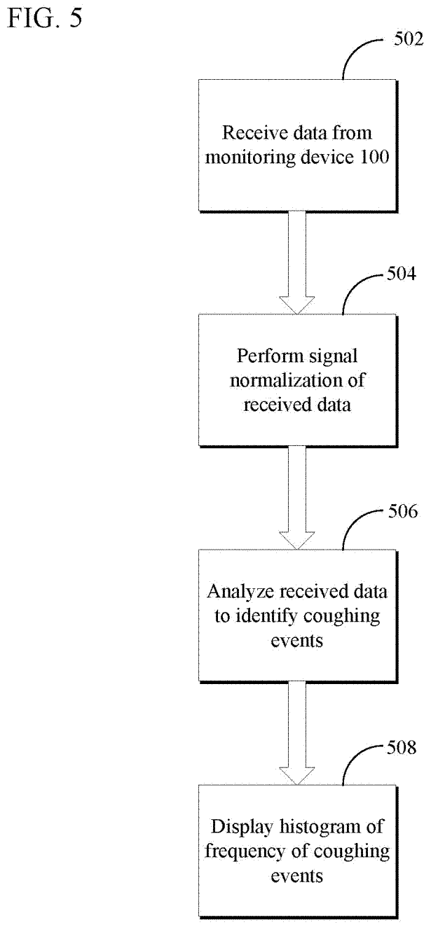

FIG. 5 is a flow chart for analyzing data for determining whether or not a coughing event has occurred according to one embodiment of the invention.

At Step 502, data indicating digital voltage values is transferred from the microcontroller 202 included in the monitor device 100 to the computer device 402 wirelessly via short-range wireless communication such as Bluetooth®. In one embodiment, the data can be continuously transferred from the monitor device 100 to computer device 402 such that the computer device 402 receives the data as a continuous stream of digital voltage values and/or the data at the time of detection of a change in the digital voltage values exceeds a threshold value. Alternatively, the monitor device 100 can include memory or storage, such as a flash memory, to store the digital voltage values with specific timestamps for later syncing with the computer device 402 allowing for batch transfer of the data.

After the data is received from the monitor device 100, the computer device 402 may perform an optional step 504 of signal normalization to compensate or correct the received data. For example, signal normalization may be performed in order to correct or compensate for a patient's body mass index (BMI), waist size, movement during measurement, and/or incorrect positioning of the monitor device 100.

Additionally, when the attachment portion 104 is configured as the nylon webbing belt, tension of the belt is important for accurate readings using the sensor 206. At step 504, the computer device 402 may characterize the tautness of the belt based on the received data and perform further normalization of the data as necessary.

At Step 506, the computer device 402 analyzes the data received from the monitor device 100 to determine whether or not a coughing event has occurred.

In one embodiment, a machine learning such as support vector machine (SVM) algorithm is utilized to cluster the data received from the monitor device 100 according to the characteristics of a change in the digital voltage values and/or a duration of the change in the digital voltage values in order to identify coughing events from the data received from the monitor device 100 based on corresponding characteristics associated with each of different kinds of physiological phenomena, such as coughing, yawning, laughing, etc., thereby allowing the computer device 402 to distinguish coughing events from other phenomena.

In particular, the SVM algorithm utilizes a threshold hyperplane in a two-dimensional feature space obtained by linear regression to optimize a margin between a first class of data points in the two-dimensional feature space corresponding to physiological phenomena categorized as coughing events and a second class of data points in the two-dimensional feature space corresponding to physiological phenomena not categorized as coughing events. However, the SVM algorithm is not limited to classifying data into one of two classes, and further classes can be utilized to differentiate between each of the different kinds of the physiological phenomena above, which would result in multiple threshold hyperplanes differentiating between the physiological phenomena.

The present invention was implemented using a semi-supervised SVM algorithm based on test datasets and training datasets obtained as a result of initial testing the monitor device 100. However, it should be understood that the present invention is not limited in such a manner and that the SVM algorithm may be implemented unsupervised or fully supervised with a larger training dataset.

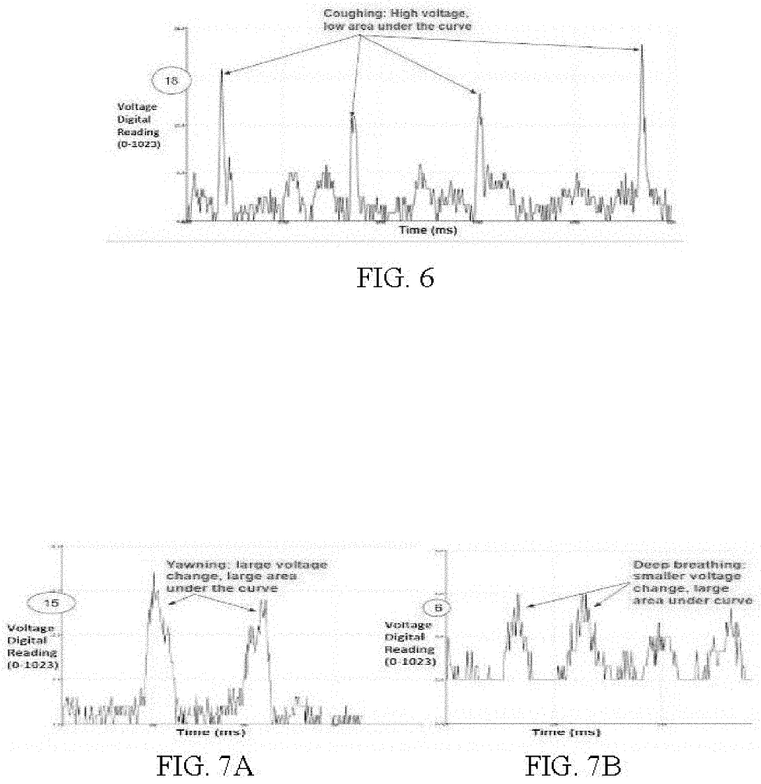

FIG. 6 illustrates a graph depicting digital voltage values obtained during a coughing event during the initial testing of the monitor device 100.

As can be seen from FIG. 6, a coughing event produces a rapid increase and subsequent rapid decrease in digital voltage values obtained by the monitor device 100, that is, when graphed, a coughing event produces a high peak digital voltage value along with a low area under a curve representing the coughing event. Accordingly, the testing results reveal that a coughing event is characterized by a significant change in digital voltage values over a short duration.

On the other hand, FIGS. 7A and 7B illustrate graphs depicting digital voltage values obtained during a yawning event and a deep breathing event, respectively, during the initial testing of the monitor device 100.

As can be seen from FIG. 7A, a yawning event produces a large and sustained increase in digital voltage values obtained by the monitor device 100, that is, when graphed, a yawning event produces a large change in digital voltage value along with a large area under a curve representing the yawning event.

Similarly, as can be seen from FIG. 7B, a deep breathing event produces a small and sustained increase in digital voltage values obtained by the monitor device 100, that is, when graphed, a deep breathing event produces a small change in digital voltage value along with a large area under a curve representing the deep breathing event.

Directly comparing the graphs illustrated in FIGS. 6, 7A, and 7B, it can be seen that the instantaneous expansion of the lungs prior to a cough and the subsequent contraction results in a distinctive change in the digital voltage values, as illustrated in FIG. 6, which is sufficiently different from that which is caused by other phenomena, such as illustrated in FIGS. 7A and 7B. Accordingly, based on these differences, the data received from the monitoring device 100 can be represented in a two-dimensional feature space and categorized according to the SVM algorithm. Data categorized as a coughing event can be stored in a memory of the computer device 402, and subsequently recalled to generate measures of frequency of coughing events.

Based on the results of the testing, the initial testing data was partitioned into feature points in the two-dimensional feature space and categorized according to the physiological phenomena present in order to create test datasets and training datasets representing the first class of data points corresponding to physiological phenomena categorized as coughing events and the second class of data points corresponding to physiological phenomena not categorized as coughing events in order to optimize the SVM algorithm utilized at Step 506.

The initial testing data may be partitioned into the features points in many different ways, for example, a given dataset could be partitioned into a two-dimensional feature point representing a first numerical feature corresponding to a magnitude of a change in voltage of the digital voltage values and a second numerical feature representing a duration from when the voltage increases and subsequently returns back to an initial value. It should be understood that the SVM algorithm is not limited to a two-dimensional feature space, and n-dimensional feature vectors representing datasets in a n-dimensional feature space may be used.

The labeling of these datasets enabled the training of the SVM algorithm and allowed for initial measurements of accuracy to estimate the sensitivity and specificity that can be expected for the cough detection system 400. Initial testing after the training of the SVM algorithm resulted in just a 1% error in identification and categorization of coughing events, thereby demonstrating improvement in cough detection by the present invention over the previously described conventional techniques.

Based on the trained SVM algorithm described above, the computer device 402 is able to, at Step 506, differentiate and identify a coughing event from other physiological phenomena by partitioning the data received from the monitor device 100 into feature points in the two-dimensional feature space based on the characteristics of a change in magnitude of the digital voltage values and/or a duration of the change in magnitude of the digital voltage values indicated by the data and categorizing the feature points according to the threshold hyperplane obtained by training the SVM algorithm.

Training of the SVM algorithm may be updated based on identified coughing events such that feature points determined to identify a coughing event may added to the first class of data points in the two-dimensional feature space corresponding to physiological phenomena categorized as coughing events, whereas feature points determined not to identify a coughing event may be added to the second class of data points in the two-dimensional feature space corresponding to physiological phenomena not categorized as coughing events, thereby allowing the SVM algorithm to update the threshold hyperplane based on new data obtained from the monitoring device 100.

Additionally, the initial testing data provided crucial information on the placement of the monitor device 100, and in particular, after testing the monitoring device 100 on the lower and mid-abdominals, it was found that placing the monitoring device 100 so that the sensor 206 is located on the ribs of the patient, specifically the eighth or ninth intercostal space near the costal cartilage, provided the clearest data. Further, it was also discovered, that clearest data is obtained by the monitoring device 100 when the attachment portion 104 has very low elasticity, thereby resulting in the attachment portion 104 being implemented as a nylon webbing belt in the present invention. The computer device 402 may utilize this initial testing data during the optional Step 504 of signal normalization to compensate or correct the received data.

In order to reduce the need for signal normalization at Step 504, clinicians may first place the monitoring device 100 on the patient under the guidance of carefully crafted instructions, thereby increasing the likelihood that the monitoring device 100 is in the optimal location for sensing the expansion of the lungs and the subsequent contraction associated with coughing and that the monitoring device 100 is also comfortable for the patient.

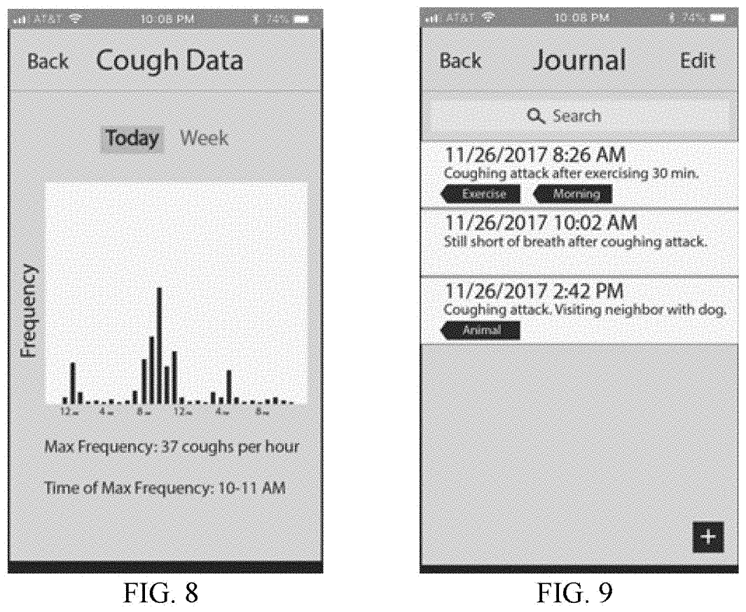

At Step 508, the computer device 402 will present the analyzed data received from the monitoring device 100 through the graphical representation of a frequency of coughing events via a display so that it may be viewed by the patient or the patient's doctor. FIG. 8 illustrates one example of display of the frequency of coughing events as a histogram. Communicating the analyzed objective data gathered from the device along with the subjective input provided by the patient in real-time is imperative for the data to be most useful in monitoring the severity of chronic cough and the efficacy of prescribed treatments. It should be readily understood that output of the analyzed data is not limited to the example illustrated in FIG. 8, and that one of ordinary skill in the art would be able to adapt the output of the analyzed data as necessary based on a particular need or desire of the patient or doctor.

As described above, data indicating digital voltage values is transferred from the monitoring device 100 to the computer device 402 for analysis. However, in another embodiment, the analysis functions performed by the computer device 402 may be implemented by the microcontroller 202 included in the monitoring device 100 such that only the results of the analysis are communicated from the monitoring device 100 to the computer device 402 for display via the computer program executed by the computer device 402.

As noted above, the computer device 402 may be configured as a smartphone. In such a configuration, the computer program executed by the computer device 402 may take the form of a smartphone application (“app”) and the computer device 402 may be configured to be able to correlate other health data with the frequency of coughing events to provide a better overall idea of the patient's daily health patterns. In other words, a context for the frequency of coughing events can be provided by integrating the functions of the present invention with data from other wearable sensors, such as number of steps/distance traveled obtained from a wearable device that functions as a pedometer, real-time heart rate from a wearable device that functions as a heart-rate monitor, etc.

Another embodiment of the invention allows the objective measurements of coughing events by the cough detection system 400 with subjective feedback from the patient. Specifically, the computer device 402 is configured to allow patient input using the executed computer program at the time of a coughing event, as illustrated in FIG. 9.

For example, if the patient begins to cough while exercising, the patient could open the computer program on the computer device 402, input the coughing event, and label the coughing event with appropriate keywords so as to indicate that the coughing event is correlated with exercising. FIG. 9 illustrates, for example, that the patient has inputted a coughing event on Nov. 26, 2017 at 8:26 AM, and that labeled the coughing event with the keywords “Exercise” and “Morning.”

Additionally, by identifying a coughing event, the cough detection system 400 may be able to combine the subjective feedback from the patient with characteristics of the digital voltage values at the time of the patient feedback in order to update the SVM algorithm.

By receiving input from the patient identifying a coughing even at a given time, the cough detection system 400 is able to retrieve data indicating characteristics of a change in the digital voltage values and/or a duration of the change in the digital voltage values received form the monitoring device 100 at the given time, create a feature point in the two-dimensional feature space using the retrieved data, and add the newly created feature point to the first class of data points in the two-dimensional feature space corresponding to physiological phenomena categorized as coughing events, thereby allowing the SVM algorithm to update the threshold hyperplane based on input from the patient.

In addition to being able to label coughing events with keywords, a patient can also keep a journal consisting of any and all notes the patient thinks might be helpful to a pulmonologist. Specifically, the patient could discuss use of the cough detection system 400 with a pulmonologist, and the pulmonologist will be able to give patients examples of pertinent information they would like noted. By using the cough detection system 400 in such a manner, both the objective measurements of coughing events by the cough detection system 400 with subjective feedback from the patient are able to provide the pulmonologist with more reliable when making a diagnosis or monitoring the success of a treatment than current techniques.

As described above, the cough detection system 400 has demonstrated an improvement over conventional technique related to identification of a coughing event from among other physiological phenomena. Further, as described above, the principles of design for the monitoring device 100 of the cough detection system 400 are based around patient comfort, and as such, allow the monitoring device 100 to be used for long periods of time, e.g., 24 hour periods, and allow the monitoring device 100 to be easily used by the patient, thereby increasing patient compliance with prescribed use by a doctor.

The present disclosure includes the use of computer programs or algorithms. The programs or algorithms can be stored on a non-transitory computer-readable medium for causing a computer, such as the one or more processors, to execute the steps described in FIG. 5. For example, the one or more memories stores software or algorithms with executable instructions and the one or more processors can execute a set of instructions of the software or algorithms in association with executing generating and processing provisioning and re-provisioning messages, and provisioning and re-provisioning responses, as described in FIG. 5.

The computer programs, which can also be referred to as programs, software, software applications, applications, components, or code, include machine instructions for a programmable processor, and can be implemented in a high-level procedural language, an object-oriented programming language, a functional programming language, a logical programming language, or an assembly language or machine language. The term computer-readable recording medium refers to any computer program product, apparatus or device, such as a magnetic disk, optical disk, solid-state storage device, memory, and programmable logic devices (PLDs), used to provide machine instructions or data to a programmable data processor, including a computer-readable recording medium that receives machine instructions as a computer-readable signal.

By way of example, a computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, include compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

Use of the phrases “capable of,” “capable to,” “operable to,” or “configured to” in one or more embodiments, refers to some apparatus, logic, hardware, and/or element designed in such a way to enable use of the apparatus, logic, hardware, and/or element in a specified manner.

The subject matter of the present disclosure is provided as examples of apparatus, systems, methods, and programs for performing the features described in the present disclosure. However, further features or variations are contemplated in addition to the features described above. It is contemplated that the implementation of the components and functions of the present disclosure can be done with any newly arising technology that may replace any of the above implemented technologies.

Additionally, the above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

1. A method for identifying a coughing event, the method comprising:
obtaining, from a sensor included in a monitoring device worn by a patient, a plurality of digital voltage values; and
analyzing the plurality of digital voltage values to identify a physiological event based on (i) a change in magnitude of the digital voltage values and (ii) a duration of the change in the magnitude of the digital voltage values, the change in the magnitude of the digital voltage values and the duration of the change in the magnitude of the digital voltage values representing a first point in a two-dimensional feature space; and
categorizing the identified physiological event as the coughing event based on a threshold hyperplane existing in the two-dimensional feature space.

2. The method for identifying the coughing event according to claim 1, wherein the threshold hyperplane is obtained by linear regression to optimize a margin between (i) a first class of one or more points in the two-dimensional feature space corresponding to a physiological event that is a coughing event and (ii) a second class of one or more points in the two-dimensional feature space corresponding to a physiological event that is not a coughing event.

3. The method for identifying the coughing event according to claim 2, wherein the threshold hyperplane is updated based on a result of categorizing the identified physiological event as the coughing event such that (i) when the identified physiological event is categorized as the coughing event, the first point in the two-dimensional feature space is added to the first class of the one or more points in the two-dimensional feature space and (ii) when the identified physiological event is not categorized as the coughing event, the first point in the two-dimensional feature space is added to the second class of one or more points in the two-dimensional feature space.

4. The method for identifying the coughing event according to claim 2, further comprising:
receiving input from the patient identifying a coughing event at an inputted time;
creating, using digital voltage values from among the plurality of digital voltage values corresponding to the inputted time, a point in the two-dimensional feature space; and
updating threshold hyperplane to add the created point to the first class of the one or more points in the two-dimensional feature space.

5. The method for identifying the coughing event according to claim 1, further comprising when the identified physiological event is categorized as the coughing event, outputting an indication of the coughing event to a display.

6. The method for identifying the coughing event according to claim 1, further comprising:
when the identified physiological event is categorized as the coughing event, storing data representing a time at which the coughing event occurred; and
outputting, to a display, a graph representing a frequency of coughing events based on the stored data.

7. The method for identifying the coughing event according to claim 1, wherein the sensor is a piezoelectric sensor configured to output a voltage value in response to strain placed upon the piezoelectric sensor by a patient as a result of a physiological event.

8. An apparatus for identifying a coughing event, the apparatus comprising:
a processor; and
a non-transitory memory having stored thereon executable instructions, which when executed cause the processor to perform operations including:
obtaining, from a sensor included in a monitoring device worn by a patient, a plurality of digital voltage values;
analyzing the plurality of digital voltage values to identify a physiological event based on (i) a change in magnitude of the digital voltage values and (ii) a duration of the change in the magnitude of the digital voltage values, the change in the magnitude of the digital voltage values and the duration of the change in the magnitude of the digital voltage values representing a first point in a two-dimensional feature space; and
categorizing the identified physiological event as the coughing event based on a threshold hyperplane existing in the two-dimensional feature space.

9. The apparatus for identifying the coughing event according to claim 8, wherein the threshold hyperplane is obtained by linear regression to optimize a margin between (i) a first class of one or more points in the two-dimensional feature space corresponding to a physiological event that is a coughing event and (ii) a second class of one or more points in the two-dimensional feature space corresponding to a physiological event that is not a coughing event.

10. The apparatus for identifying the coughing event according to claim 9, wherein the threshold hyperplane is updated based on a result of categorizing the identified physiological event as the coughing event such that (i) when the identified physiological event is categorized as the coughing event, the first point in the two-dimensional feature space is added to the first class of the one or more points in the two-dimensional feature space and (ii) when the identified physiological event is not categorized as the coughing event, the first point in the two-dimensional feature space is added to the second class of one or more points in the two-dimensional feature space.

11. The apparatus for identifying the coughing event according to claim 9, wherein the executable instructions further cause the processor to perform operations including:
receiving input from the patient identifying a coughing event at an inputted time;
creating, using digital voltage values from among the plurality of digital voltage values corresponding to the inputted time, a point in the two-dimensional feature space; and
updating threshold hyperplane to add the created point to the first class of the one or more points in the two-dimensional feature space.

12. The apparatus for identifying the coughing event according to claim 8, wherein the executable instructions further cause the processor to perform an operation of when the identified physiological event is categorized as the coughing event, outputting an indication of the coughing event to a display.

13. The apparatus for identifying the coughing event according to claim 8, wherein the executable instructions further cause the processor to perform operations including:
when the identified physiological event is categorized as the coughing event, storing data representing a time at which the coughing event occurred; and
outputting, to a display, a graph representing a frequency of coughing events based on the stored data.

14. The apparatus for identifying the coughing event according to claim 8, wherein the sensor is a piezoelectric sensor configured to output a voltage value in response to strain placed upon the piezoelectric sensor by a patient as a result of a physiological event.

15. A system for identifying a coughing event, the system comprising:
a monitoring device including a microcontroller and a piezoelectric sensor configured to output voltage values to the microcontroller in response to strain placed upon the piezoelectric sensor by a patient as a result of a physiological event, the microcontroller being capable of performing short-range wireless communication; and
a computer device capable of performing the short-range wireless communication,
wherein microcontroller converts the voltage values obtained from the piezoelectric sensor to digital voltage values, and outputs the digital voltage values to the computer device using the short-range wireless communication,
wherein the computer device analyzes the plurality of digital voltage values to identify a physiological event based on (i) a change in magnitude of the digital voltage values and (ii) a duration of the change in the magnitude of the digital voltage values, the change in the magnitude of the digital voltage values and the duration of the change in the magnitude of the digital voltage values representing a first point in a two-dimensional feature space,
wherein the threshold hyperplane is obtained by linear regression to optimize a margin between (i) a first class of one or more points in the two-dimensional feature space corresponding to a physiological event that is a coughing event and (ii) a second class of one or more points in the two-dimensional feature space corresponding to a physiological event that is not a coughing event,
wherein the computer device categorizes the identified physiological event as the coughing event based on a threshold hyperplane existing in the two-dimensional feature space, and
wherein when the identified physiological event is categorized as the coughing event, the computer device outputs an indication of the coughing event to a display.
